(19) United States
(12) Patent Application Publication
(10) Pub. No.: US 2026/0070859 A1
(43) Pub. Date: Mar. 12, 2026

(54) METHOD FOR INCREASING PRODUCT RECOVERY IN AN OLIGOMERIZATION PROCESS

(71) Applicant: UOP LLC, Des Plaines, IL (US)

(72) Inventors: Jeannie M. Blommel, Oregon, WI (US); Matthew C. Cole, Evanston, IL (US); Joel S. Paustian, Vernon Hills, IL (US); Manuela Serban, Northbrook, IL (US)

(21) Appl. No.: 18/826,477

(22) Filed: Sep. 6, 2024

(51) Int. Cl. C07C 2/12; C07C 5/03

(52) U.S. Cl. CPC C07C 2/12; C07C 5/03; C07C 2529/06

(57) ABSTRACT

Methods for increasing product recovery in an oligomerization process are described. The methods comprise incorporating an additional oligomerization reaction zone to oligomerize a portion of the olefin splitter bottom stream and/or the depropanizer overhead stream. The effluent from the additional oligomerization reaction zone is combined with the olefin splitter bottom stream, hydrogenated and fractionated.

### BACKGROUND

Oligomerization processes can be used to oligomerize C₂ to C₈ olefins into aviation and diesel fuels. The feed includes a small amount of light paraffins, byproducts are formed in the oligomerization reactions. To prevent the accumulation of the light paraffins, unconverted ethylene and propylene are fractionated in a depropanizer column as an off gas stream and a small drag stream from the light olefin recycle is sent to the hydrogenation reactor to be saturated. The saturated components from the drag stream become fuel gas and naphtha product in the downstream fractionation section. Customers typically want to maximize sustainable aviation fuel production and minimize byproducts. A carbon balance in a methanol to jet oligomerization unit shows about 3000 kg/hr of carbon in the depropanizer off gas stream and about 4000 kg/hr in the drag stream. There are approximately 30-50 wt% olefins in the depropanizer off gas stream and 50-70 wt% olefins in the drag stream. Of the olefins in the drag stream, approximately 700-800 kg/hr of it goes into the jet product already. Theoretically, there is potential to recover as much as 2000-3000 kg/hr of the carbon lost. Ethylene to jet oligomerization has similar carbon losses, although the drag stream is normally smaller for similar sized unit.

Therefore, there is a need for a process for increasing product recovery and reducing carbon losses in an oligomerization process.

### BRIEF DESCRIPTION OF THE DRAWINGS

FIG. 1 is an illustration of one embodiment of the process of the present invention.

### DESCRIPTION

The present invention accomplishes this goal by incorporating a small oligomerization reactor in the oligomerization process. The new reactor can oligomerize the olefins within the drag stream and/or the depropanizer off gas. If processing the depropanizer off gas is desired, a small compressor can be added to increase the off gas pressure to the oligomerization operating pressure. The drag stream is already at sufficient pressure for the oligomerization reactor. The catalyst in the new reactor may comprise a zeolitic catalyst, a layer of a zeolitic catalyst and a layer of a metal catalyst, or a mixture of a zeolitic catalyst and a metal catalyst. Assuming 95% of the olefins in the streams oligomerize to naphtha, jet, and distillate products and with the same distribution, there would be an additional 2000-2500 kg/hr of jet and 30-50 kg/hr of diesel blendstock, a 3-10% increase. The new reactor can be oversized such that it can be manually regenerated in a lower frequency than the main reactors. If planned properly, it would have minimal impact to the regeneration requirements of the main reactors. It can have a spare or not, depending on the economics.

One aspect of the present invention comprises a method for increasing product recovery in an oligomerization process.

In one embodiment, the method comprises oligomerizing a mixed olefin stream comprising C₂₋₈ olefins in an oligomerization reaction zone comprising an oligomerization reactor in the presence of an oligomerization catalyst to form an oligomerization effluent stream comprising C₉₊ olefins and unreacted C₂₋₈ olefins.

The oligomerization catalyst can be any suitable oligomerization catalyst that will catalyze the reaction of C₂₋₈ olefins to C₉₊ olefins under the reaction conditions being used. Suitable oligomerization catalysts include, but are not limited to, zeolitic catalysts, a layer of a zeolitic catalyst and a layer of a metal catalyst, or a mixture of a zeolitic catalyst and a metal catalyst.

The oligomerization reaction conditions include a temperature in the range of 100° C. to 300° C., and a pressure in the range of 3.5 MPa to 7.0 MPa.

The oligomerization effluent stream is separated into a depropanizer overhead stream comprising unconverted C₂₋₄ olefins and a depropanizer bottom stream comprising C₄₊ olefins in a depropanizer column.

The oligomerization reaction zone may comprise one or more reactors. The oligomerization reaction can be performed in one stage or more than one stage, e.g., two stages, three stages, etc. The oligomerization reaction zone may comprise one reactor with one stage of oligomerization, one reactor with two stages of oliomerization, two reactors in which the first reactor performs the first stage of oligomerization and a second reactor which performs the second stage. Each of the one or more reactors can perform one or more stages of the oligomerization reaction. Those of skill in the art can design an appropriate reaction zone for the particular circumstances of the process.

The depropanizer bottom stream is separated into an olefin splitter overhead stream comprising C₈₋ olefins and an olefin splitter bottom stream comprising C₉₊ olefins in an olefin splitter column.

The olefin splitter overhead stream is condensed into an olefin splitter liquid stream comprising C₄₋₈ olefins.

The process comprises a drag oligomerization reaction zone comprising a drag oligomerization reactor. There can be one or more reactors, and one or more stages in the drag oligomerization reaction zone, as discussed above. The drag oligomerization reaction conditions and catalysts may be those discussed above.

A first portion of the olefin splitter liquid stream and/or the depropanizer overhead stream is oligomerized in the drag oligomerization reaction zone to form the drag oligomerization effluent stream comprising C₉₊ olefins. A diluent stream may be introduced into the drag oligomerization reaction zone to control the oligomerization reaction exotherm.

The olefin splitter bottom stream and the drag oligomerization effluent stream are hydrogenated in a hydrogenation reaction zone comprising a hydrogenation reactor to form a hydrogenation effluent stream comprising C₉₊ paraffins.

The hydrogenation catalyst can be any suitable hydrogenation catalyst that will catalyze the reaction of olefins to paraffins under the reaction conditions being used. Suitable hydrogenation catalysts include, but are not limited to, a noble metal catalyst, such as platinum, on a support, such as an alumina support.

The hydrogenation reaction conditions include a temperature in the range of 170° C. to 300° C., and a pressure in the range of 2.5 MPa and 5.0 MPa.

The hydrogenation effluent stream is separated in a jet fractionation section comprising a fractionation column into at least a synthetic paraffinic kerosene stream comprising C₉₋₁₉ paraffins, and a diesel blendstock stream comprising C₁₉₊ paraffins.

As is known in the art, separation processes are not perfect, and small amounts of hydrocarbons outside a range may also be included in a stream. For example, the synthetic paraffinic kerosene stream comprising C₉₋₁₉ paraffins may also include some C₈ and C₂₀ paraffins, as long as it meets ASTM D7566 specifications (including the flash point and end point specifications).

In some embodiments, the method further comprises stripping the hydrogenation effluent stream in a flash stripper to form a flash stripper overhead stream comprising C5-hydrocarbons and hydrogen and a flash stripper bottom stream comprising C₆₊ paraffins. In this case, separating the hydrogenation effluent stream comprises separating the flash stripper bottom stream. The flash stripper overhead stream may be routed to the fuel gas system and burned in fired heaters, for example.

In some embodiments, the method further comprises compressing the depropanizer overhead stream. In this situation, oligomerizing the depropanizer overhead stream comprises oligomerizing the compressed depropanizer overhead stream.

In some embodiments, the method further comprises dividing the olefin splitter liquid stream into the first portion and a second portion and recycling the second portion to the oligomerization reaction zone.

In some embodiments, separating the hydrogenation effluent stream comprises separating the hydrogenation effluent stream into at least the synthetic paraffinic kerosene stream, the diesel blendstock stream, a naphtha stream comprising C₅₋₈ paraffins, and optionally a jet fraction off-gas stream comprising C₅₋ paraffins and hydrogen.

In some embodiments, the mixed olefin stream comprises the product of an alcohol to aviation fuel process.

In some embodiments, the method further comprises refluxing a portion of the olefin splitter liquid stream to the olefin splitter column.

Another aspect of the invention is a method for increasing product recovery in an oligomerization process. In one embodiment, the method comprises oligomerizing a mixed olefin stream comprising C₂₋₈ olefins in an oligomerization reaction zone comprising an oligomerization reactor in the presence of an oligomerization catalyst to form an oligomerization effluent stream comprising C₉₊ olefins and unreacted C₂₋₈ olefins.

The oligomerization effluent stream is separated into a depropanizer overhead stream comprising unconverted C₂₋₄ olefins and a depropanizer bottom stream comprising C₄₊ olefins in a depropanizer column.

The depropanizer bottom stream is separated into an olefin splitter overhead stream comprising C₈₋ olefins and an olefin splitter bottom stream comprising C₉₊ olefins in an olefin splitter column.

The olefin splitter overhead stream is condensed into an olefin splitter liquid stream comprising C₄₋₈ olefins, and the condensed olefin splitter liquid stream is divided into a recycle stream and a drag stream. The recycle stream is recycled to the oligomerization reaction zone.

The drag stream and/or the depropanizer overhead stream is oligomerized in a drag oligomerization reaction zone comprising a drag oligomerization reactor in the presence of a drag oligomerization catalyst to form a drag oligomerization effluent stream comprising C₉₊ olefins.

The olefin splitter bottom stream and the drag oligomerization effluent stream are hydrogenated in a hydrogenation reaction zone comprising a hydrogenation reactor to form a hydrogenation effluent stream comprising C₉₊ paraffins.

The hydrogenation effluent stream is stripped in a flash stripper to form a flash stripper overhead stream comprising C₅₋ hydrocarbons and hydrogen and a flash stripper bottom stream comprising C₆₊ paraffins.

The flash stripper bottom stream is separated in a jet fractionation section comprising a fractionation column into at least a synthetic paraffinic kerosene stream comprising approximately C₉₋₁₉ paraffins, and a diesel blendstock stream comprising C₁₉₊ paraffins. There can be small amounts C₈ and C₂₀ paraffins in the synthetic paraffinic kerosene stream as long as it meets the ASTM D7566 specifications. In some embodiments, the method further comprises compressing the depropanizer overhead stream. In this case, oligomerizing the depropanizer overhead stream comprises oligomerizing the compressed depropanizer overhead stream.

In some embodiments, separating the hydrogenation effluent stream comprises separating the hydrogenation effluent stream into at least the synthetic paraffinic kerosene stream, the diesel blendstock stream, a naphtha stream comprising C₅₋₈ paraffins, and optionally a jet fraction off-gas stream comprising C₅₋ paraffins and hydrogen.

In some embodiments, the mixed olefin stream comprises the product of an alcohol to aviation fuel process.

The oligomerization catalyst and/or the drag oligomerization catalyst may be as discussed above.

The oligomerization reaction zone and/or the drag oligomerization reaction zone may comprises one or more reactors and one or more stages as discussed above.

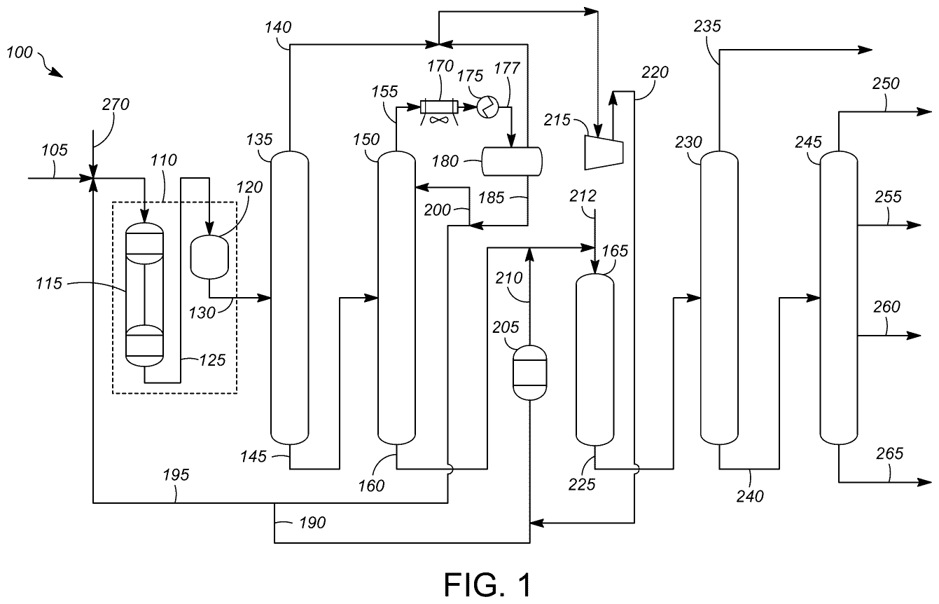

FIG. 1 illustrates one embodiment of a process 100 according to the present invention. A mixed olefin stream 105 is sent to the oligomerization reaction zone 110. The mixed olefin stream 105 may comprise C₂₋₈ olefins. The mixed olefin stream 105 may be from an alcohol to olefin process, for example.

As shown, the oligomerization reaction zone 110 includes first stage oligomerization reactor 115 and second stage oligomerization reactor 120. The effluent stream 125 from the first stage oligomerization reactor 115 is sent to the second stage oligomerization reactor 120.

The oligomerization effluent stream 130 from the second stage oligomerization reactor 120 comprises C₉₊ olefins and unreacted C₂₋₈ olefins. The oligomerization effluent stream 130 is sent to the depropanizer column 135 where it is separated into a depropanizer overhead stream 140 comprising unconverted C₂₋₄ olefins and a depropanizer bottom stream 145 comprising C₄₊ olefins.

The depropanizer bottom stream 145 is sent to the olefin splitter column 150 where it is separated into an olefin splitter overhead stream 155 comprising C₈₋ olefins and an olefin splitter bottom stream 160 comprising C₉₊ olefins.

The olefin splitter overhead stream 155 is cooled in air cooler 170 and heat exchanger 175, and the condensed stream 177 is sent to the olefin splitter receiver 180. The olefin splitter liquid stream 185 from the olefin splitter receiver 180 can be divided into multiple liquid streams, for example, a drag stream 190 and a recycle stream 195. In addition, a portion 200 of the olefin splitter liquid stream 185 can be refluxed to the olefin splitter column 150.

The recycle stream 195 can be recycled to the oligomerization reaction zone 110.

The drag stream 190 can be sent to a drag oligomerization reactor 205 where the olefins are oligomerized. A small diluent stream (not shown) may be added to the drag oligomerization reactor 205 to manage the oligomerization reaction exotherm. The diluent may comprise C₉₊ paraffins, for example.

The olefin splitter bottom stream 160 comprising C₉₊ olefins and the drag reactor effluent stream 210 comprising C₉₊ olefins are sent to the hydrogenation reactor 165 along with hydrogen makeup stream 212 where the olefins are converted to C₉₊ paraffins.

The depropanizer overhead stream 140 comprising unconverted C₂₋₄ olefins can be sent to a compressor 215. The compressed olefin splitter overhead stream 220 can be sent to the drag oligomerization reactor 205 where the olefins are oligomerized.

The hydrogenation reactor effluent stream 225 is sent to the flash stripper column 230 where it is separated into a flash stripper overhead stream 235 and a flash stripper bottom stream 240. The flash stripper overhead stream 235 comprising C₅₋ hydrocarbons is sent to the fuel gas system and burned in fired heaters, for example.

The flash stripper bottom stream 240 comprising C₆₊ paraffins is sent to a jet fractionation section comprising a fractionation column 245 where it is separated into an off-gas stream 250, a renewable naphtha stream 255, a synthetic paraffinic kerosene stream 260, and a diesel blendstock stream 265. The off-gas stream 250 comprises C₇₋ paraffins and hydrogen and is routed to the fuel gas system and burned in the fired heaters, for example. The renewable naphtha stream 255 comprises C₅₋₈ paraffins and can be stabilized and used as gasoline blending stock, as a feedstock to steam cracking processes and the like, or pumped to a fired heater as fuel. The synthetic paraffinic kerosene stream 260 comprises C₉₋₁₉ paraffins and may be blended and used as aviation fuel. The diesel blendstock stream 265 comprises C₁₉₊ paraffins and may be used as diesel blendstock. Those of skill in the art will understand that other separations could be performed.

A portion 270 of the hydrogenation reactor effluent stream 225 from the hydrogenation reactor 165 can be recycled to the oligomerization reaction zone 110 as recycle oil stream 270, which comprises C₉₊ paraffins. It can be mixed with the mixed olefin stream 105 and the recycle stream 195 from the olefin splitter receiver 180.

### SPECIFIC EMBODIMENTS

While the following is described in conjunction with specific embodiments, it will be understood that this description is intended to illustrate and not limit the scope of the preceding description and the appended claims.

A first embodiment of the invention is a method for increasing product recovery in an oligomerization process comprising oligomerizing a mixed olefin stream comprising C₂₋₈ olefins in an oligomerization reaction zone comprising an oligomerization reactor in the presence of an oligomerization catalyst to form an oligomerization effluent stream comprising C₉₊ olefins and unreacted C₂₋₈ olefins; separating the oligomerization effluent stream in a depropanizer column into a depropanizer overhead stream comprising unconverted C₂₋₄ olefins and a depropanizer bottom stream comprising C₄₊ olefins; separating the depropanizer bottom stream in an olefin splitter column into an olefin splitter overhead stream comprising C₈₋ olefins and an olefin splitter bottom stream comprising C₉₊ olefins; condensing the olefin splitter overhead stream into an olefin splitter liquid stream comprising C₄₋₈ olefins; oligomerizing a first portion of the olefin splitter liquid stream in a drag oligomerization reaction zone comprising a drag oligomerization reactor in the presence of a drag oligomerization catalyst to form a drag oligomerization effluent stream comprising C₉₊ olefins, or oligomerizing the depropanizer overhead stream in the drag oligomerization reaction zone comprising the drag oligomerization reactor in the presence of the drag oligomerization catalyst to form the drag oligomerization effluent stream comprising C₉₊ olefins, or both; hydrogenating the olefin splitter bottom stream and the drag oligomerization effluent stream in a hydrogenation reaction zone comprising a hydrogenation reactor to form a hydrogenation effluent stream comprising C₉₊ paraffins; separating the hydrogenation effluent stream in a jet fractionation section comprising a fractionation column into at least a synthetic paraffinic kerosene stream comprising C₉₋₁₉ paraffins and a diesel blendstock stream comprising C₁₉₊ paraffins. An embodiment of the invention is one, any or all of prior embodiments in this paragraph up through the first embodiment in this paragraph further comprising stripping the hydrogenation effluent stream in a flash stripper to form a flash stripper overhead stream comprising C₅₋ hydrocarbons and hydrogen and a flash stripper bottom stream comprising C₆₊ paraffins; and wherein separating the hydrogenation effluent stream comprises separating the flash stripper bottom stream. An embodiment of the invention is one, any or all of prior embodiments in this paragraph up through the first embodiment in this paragraph further comprising compressing the depropanizer overhead stream; and wherein oligomerizing the depropanizer overhead stream comprises oligomerizing the compressed depropanizer overhead stream. An embodiment of the invention is one, any or all of prior embodiments in this paragraph up through the first embodiment in this paragraph further comprising dividing the olefin splitter liquid stream into the first portion and a second portion; and recycling the second portion to the oligomerization reaction zone. An embodiment of the invention is one, any or all of prior embodiments in this paragraph up through the first embodiment in this paragraph wherein separating the hydrogenation effluent stream comprises separating the hydrogenation effluent stream into at least the synthetic paraffinic kerosene stream, the diesel blendstock stream, a naphtha stream comprising C₅₋₈ paraffins, and optionally a jet fraction off-gas stream comprising C₅₋ paraffins and hydrogen. An embodiment of the invention is one, any or all of prior embodiments in this paragraph up through the first embodiment in this paragraph wherein the mixed olefin stream comprises the product of an alcohol to olefin process. An embodiment of the invention is one, any or all of prior embodiments in this paragraph up through the first embodiment in this paragraph further comprising refluxing a portion of the olefin splitter liquid stream to the olefin splitter column. An embodiment of the invention is one, any or all of prior embodiments in this paragraph up through the first embodiment in this paragraph wherein the oligomerization catalyst or the drag oligomerization catalyst or both comprises a zeolitic catalyst, a layer of a zeolitic catalyst and a layer of a metal catalyst, or a mixture of a zeolitic catalyst and a metal catalyst. An embodiment of the invention is one, any or all of prior embodiments in this paragraph up through the first embodiment in this paragraph wherein the oligomerization reaction zone comprises at least two stages. An embodiment of the invention is one, any or all of prior embodiments in this paragraph up through the first embodiment in this paragraph wherein the at least two stages are in separate reactors. An embodiment of the invention is one, any or all of prior embodiments in this paragraph up through the first embodiment in this paragraph wherein the at least two stages are in a single reactor.

A second embodiment of the invention is a method for increasing product recovery in an oligomerization process comprising oligomerizing a mixed olefin stream comprising C₂₋₈ olefins in an oligomerization reaction zone comprising an oligomerization reactor in the presence of an oligomerization catalyst to form an oligomerization effluent stream comprising C₉₊ olefins and unreacted C₂₋₈ olefins; separating the oligomerization effluent stream in a depropanizer column into a depropanizer overhead stream comprising unconverted C₂₋₄ olefins and a depropanizer bottom stream comprising C₄₊ olefins; separating the depropanizer bottom stream in an olefin splitter column into an olefin splitter overhead stream comprising C₈₋ olefins and an olefin splitter bottom stream comprising C₉₊ olefins; condensing the olefin splitter overhead stream into an olefin splitter liquid stream comprising C₄₋₈ olefins; dividing the condensed olefin splitter liquid stream into a recycle stream and a drag stream; recycling the recycle stream to the oligomerization reaction zone; oligomerizing the drag stream in a drag oligomerization reaction zone comprising a drag oligomerization reactor in the presence of a drag oligomerization catalyst to form a drag oligomerization effluent stream comprising C₉₊ olefins, or oligomerizing the depropanizer overhead stream in the drag oligomerization reaction zone comprising the drag oligomerization reactor in the presence of the drag oligomerization catalyst to form the drag oligomerization effluent stream comprising C₉₊ olefins, or both; hydrogenating the olefin splitter bottom stream and the drag oligomerization effluent stream in a hydrogenation reaction zone comprising a hydrogenation reactor to form a hydrogenation effluent stream comprising C₉₊ paraffins; stripping the hydrogenation effluent stream in a flash stripper to form a flash stripper overhead stream comprising C₅₋ hydrocarbons and hydrogen and a flash stripper bottom stream comprising C₆₊ paraffins; separating the flash stripper bottom stream in a jet fractionation section comprising a fractionation column into at least a synthetic paraffinic kerosene stream comprising approximately C₉₋₁₉ paraffins, meeting ASTM D7566 specifications, and a diesel blendstock stream comprising C₁₉₊ paraffins. An embodiment of the invention is one, any or all of prior embodiments in this paragraph up through the second embodiment in this paragraph further comprising compressing the depropanizer overhead stream; and wherein oligomerizing the depropanizer overhead stream comprises oligomerizing the compressed depropanizer overhead stream. An embodiment of the invention is one, any or all of prior embodiments in this paragraph up through the second embodiment in this paragraph wherein separating the hydrogenation effluent stream comprises separating the hydrogenation effluent stream into at least the synthetic paraffinic kerosene stream, the diesel blendstock stream, a naphtha stream comprising C₅₋₈ paraffins, and optionally a jet fraction off-gas stream comprising C₅₋ paraffins and hydrogen. An embodiment of the invention is one, any or all of prior embodiments in this paragraph up through the second embodiment in this paragraph wherein the mixed olefin stream comprises the product of an alcohol to olefin process. An embodiment of the invention is one, any or all of prior embodiments in this paragraph up through the second embodiment in this paragraph wherein the oligomerization catalyst or the drag oligomerization catalyst or both comprises a zeolitic catalyst, a layer of a zeolitic catalyst and a layer of a metal catalyst, or a mixture of a zeolitic catalyst and a metal catalyst. An embodiment of the invention is one, any or all of prior embodiments in this paragraph up through the second embodiment in this paragraph wherein the oligomerization reaction zone comprises at least two stages. An embodiment of the invention is one, any or all of prior embodiments in this paragraph up through the second embodiment in this paragraph wherein the at least two stages are in separate reactors. An embodiment of the invention is one, any or all of prior embodiments in this paragraph up through the second embodiment in this paragraph wherein the at least two stages are in a single reactor.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. A method for increasing product recovery in an oligomerization process comprising:
oligomerizing a mixed olefin stream comprising C₂₋₈ olefins in an oligomerization reaction zone comprising an oligomerization reactor in the presence of an oligomerization catalyst to form an oligomerization effluent stream comprising C₉₊ olefins and unreacted C₂₋₈ olefins;
separating the oligomerization effluent stream in a depropanizer column into a depropanizer overhead stream comprising unconverted C₂₋₄ olefins and a depropanizer bottom stream comprising C₄₊ olefins;
separating the depropanizer bottom stream in an olefin splitter column into an olefin splitter overhead stream comprising C₈₋ olefins and an olefin splitter bottom stream comprising C₉₊ olefins;
condensing the olefin splitter overhead stream into an olefin splitter liquid stream comprising C₄₋₈ olefins;
oligomerizing a first portion of the olefin splitter liquid stream in a drag oligomerization reaction zone comprising a drag oligomerization reactor in the presence of a drag oligomerization catalyst to form a drag oligomerization effluent stream comprising C₉₊ olefins, or oligomerizing the depropanizer overhead stream in the drag oligomerization reaction zone comprising the drag oligomerization reactor in the presence of the drag oligomerization catalyst to form the drag oligomerization effluent stream comprising C₉₊ olefins, or both;
hydrogenating the olefin splitter bottom stream and the drag oligomerization effluent stream in a hydrogenation reaction zone comprising a hydrogenation reactor to form a hydrogenation effluent stream comprising C₉₊ paraffins; and
separating the hydrogenation effluent stream in a jet fractionation section comprising a fractionation column into at least a synthetic paraffinic kerosene stream comprising C₉₋₁₉ paraffins and a diesel blendstock stream comprising C₁₉₊ paraffins.

2. The method of claim 1 further comprising:
stripping the hydrogenation effluent stream in a flash stripper to form a flash stripper overhead stream comprising C₅₋ hydrocarbons and hydrogen and a flash stripper bottom stream comprising C₆₊ paraffins; and
and wherein separating the hydrogenation effluent stream comprises separating the flash stripper bottom stream.

3. The method of claim 1 further comprising:
compressing the depropanizer overhead stream; and
wherein oligomerizing the depropanizer overhead stream comprises oligomerizing the compressed depropanizer overhead stream.

4. The method of claim 1 further comprising:
dividing the olefin splitter liquid stream into the first portion and a second portion; and
recycling the second portion to the oligomerization reaction zone.

5. The method of claim 1 wherein separating the hydrogenation effluent stream comprises separating the hydrogenation effluent stream into at least the synthetic paraffinic kerosene stream, the diesel blendstock stream, a naphtha stream comprising C₅₋₈ paraffins, and optionally a jet fraction off-gas stream comprising C₅₋ paraffins and hydrogen.

6. The method of claim 1 wherein the mixed olefin stream comprises the product of an alcohol to olefin process.

7. The method of claim 1 further comprising:
refluxing a portion of the olefin splitter liquid stream to the olefin splitter column.

8. The method of claim 1 wherein the oligomerization catalyst or the drag oligomerization catalyst or both comprises a zeolitic catalyst, a layer of a zeolitic catalyst and a layer of a metal catalyst, or a mixture of a zeolitic catalyst and a metal catalyst.

9. The method of claim 1 wherein the oligomerization reaction zone comprises at least two stages.

10. The method of claim 8 wherein the at least two stages are in separate reactors.

11. The method of claim 8 wherein the at least two stages are in a single reactor.

12. A method for increasing product recovery in an oligomerization process comprising:
oligomerizing a mixed olefin stream comprising C₂₋₈ olefins in an oligomerization reaction zone comprising an oligomerization reactor in the presence of an oligomerization catalyst to form an oligomerization effluent stream comprising C₉₊ olefins and unreacted C₂₋₈ olefins;
separating the oligomerization effluent stream in a depropanizer column into a depropanizer overhead stream comprising unconverted C₂₋₄ olefins and a depropanizer bottom stream comprising C₄₊ olefins;
separating the depropanizer bottom stream in an olefin splitter column into an olefin splitter overhead stream comprising C₈₋ olefins and an olefin splitter bottom stream comprising C₉₊ olefins;
condensing the olefin splitter overhead stream into an olefin splitter liquid stream comprising C₄₋₈ olefins;
dividing the condensed olefin splitter liquid stream into a recycle stream and a drag stream;
recycling the recycle stream to the oligomerization reaction zone;
oligomerizing the drag stream in a drag oligomerization reaction zone comprising a drag oligomerization reactor in the presence of a drag oligomerization catalyst to form a drag oligomerization effluent stream comprising C₉₊ olefins, or oligomerizing the depropanizer overhead stream in the drag oligomerization reaction zone comprising the drag oligomerization reactor in the presence of the drag oligomerization catalyst to form the drag oligomerization effluent stream comprising C₉₊ olefins, or both;
hydrogenating the olefin splitter bottom stream and the drag oligomerization effluent stream in a hydrogenation reaction zone comprising a hydrogenation reactor to form a hydrogenation effluent stream comprising C₉₊ paraffins;
stripping the hydrogenation effluent stream in a flash stripper to form a flash stripper overhead stream comprising C₅₋ hydrocarbons and hydrogen and a flash stripper bottom stream comprising C₆₊ paraffins; and
separating the flash stripper bottom stream in a jet fractionation section comprising a fractionation column into at least a synthetic paraffinic kerosene stream comprising approximately C₉₋₁₉ paraffins, meeting ASTM D7566 specifications, and a diesel blendstock stream comprising C₁₉₊ paraffins.

13. The method of claim 12 further comprising:
compressing the depropanizer overhead stream; and
wherein oligomerizing the depropanizer overhead stream comprises oligomerizing the compressed depropanizer overhead stream.

14. The method of claim 12 wherein separating the hydrogenation effluent stream comprises separating the hydrogenation effluent stream into at least the synthetic paraffinic kerosene stream, the diesel blendstock stream, a naphtha stream comprising C₅₋₈ paraffins, and optionally a jet fraction off-gas stream comprising C₅₋ paraffins and hydrogen.

15. The method of claim 12 wherein the mixed olefin stream comprises the product of an alcohol to olefin process.

16. The method of claim 12 wherein the oligomerization catalyst or the drag oligomerization catalyst or both comprises a zeolitic catalyst, a layer of a zeolitic catalyst and a layer of a metal catalyst, or a mixture of a zeolitic catalyst and a metal catalyst.

17. The method of claim 12 wherein the oligomerization reaction zone comprises at least two stages.

18. The method of claim 17 wherein the at least two stages are in separate reactors.

19. The method of claim 1 wherein the at least two stages are in a single reactor.
